(19) United States
(12) Patent Application Publication
(10) Pub. No.: US 2003/0216330 A1
(43) Pub. Date: Nov. 20, 2003

(54) Parenteral, intravenous, and oral administration of oxazolidinones for treating diabetic foot infections

(75) Inventor: Carl Norden, Wynnewood, PA (US)

Correspondence Address: MARSHALL, GERSTEIN & BORUN LLP, 6300 SEARS TOWER, 233 S. WACKER DRIVE, CHICAGO, IL, 60606, US

(73) Assignee: PHARMACIA & UPJOHN

(21) Appl. No.: 10/394,912

(22) Filed: Mar. 21, 2003

Related U.S. Application Data
(60) Provisional application No. 60/369,104, filed on Mar. 29, 2002.

(51) Int. Cl. A61K 31/7048; A61K 31/704; A61K 31/421; A61K 31/65; A61K 31/4709

(52) U.S. Cl. 514/29; 514/37; 514/380; 514/153; 514/312; 514/192

(57) ABSTRACT

A method of treating a diabetic foot infection in a mammal includes oral, parenteral, or intravenous administration of a pharmaceutical formulation containing an orally, parenterally, or intravenously-effective amount, respectively of an oxazolidinone.

### RELATED APPLICATION DATA

This application claims the benefit of U.S. provisional Application Serial No. 60/369,104 filed Mar. 29, 2002.

### FIELD OF THE INVENTION

The present invention relates to a method of treating bacterial infections. More particularly, the present invention relates to treating diabetic foot infections by parenteral, intravenous and oral administration of known pharmaceutically-useful oxazolidinone antibacterials.

### BACKGROUND OF THE INVENTION

Foot infections and their sequelae are among the most common and severe complications of diabetes mellitus (Benjamin A. Lipsky “Osteomyelitis of the Foot in Diabetic Patients” Clinical Infectious Diseases 25: 1318-1326 (1997)). Infection of the foot is the complication of diabetes that most frequently leads to hospitalization (Carl Norden et al. Infections in Bones and Joints: Part Three, p. 181 (1994)). It is a major contributing factor to diabetes being the leading cause of lower-extremity amputations in the United States The risk of lower-extremity amputation is 5- to 15-fold higher in diabetic patients, and one-half the non-traumatic amputations performed each year in the United States are in patients with diabetes. Foot bone osteomyelitis in diabetic patients is largely a consequence of contiguous infection from the overlying soft tissue. In most reports, about a third of diabetic patients with foot infections are found to have evidence of osteomyelitis. A diabetic foot infection (DFI) is an infection in the foot of a diabetic whose foot receives minor trauma in the presence of peripheral neuropathy and/or vascular disease causing the formation of an ulcer (Avery's Drug Treatment, 4^{th} ed. (1997), p. 742). Diabetic foot infections may be polymicrobial, including both aerobic and anaerobic gram positive and gram negative organisms. Aerobic gram-positive bacteria or cocci (GPC) are clearly the most common causes of these infections including but not limited to Staphylococcus aureus, group B streptococci, or Enterococci. (Lipsky, et al., “Outpatient management of uncomplicated lower-extremity infections in diabetic patients,” Arch. Intern Med 150:790-797 (1990)). These are often the sole pathogens. The important species of the staphylococci genus are Staphyloccus aureus, Staphylococcus epidermidis, and Staphylococcus hemolyticus.

Facultative gram-negative bacilli are also major pathogens in the diabetic foot infections, and often the sole pathogens. Aerobic gram-negative bacteria and anaerobes are usually recovered as part of mixed infections, especially in patients who have recently received antibiotic therapy. It is important that an antibiotic used for diabetic foot infections be effective against aerobic gram positive bacteria. It is also beneficial that a treatment cover aerobic gram-negative bacilli, enterococci, and anerobes, although infections caused solely by these organisms are more rare. Further, when bacteremia accompanies these infections it is usually caused by staphylococci or occasionally Bacteroides species. Currently, a variety of regimens is advocated for initial empiric therapy of complicated infection, and no single agent or combination has demonstrated superiority. Such infections are very difficult to treat with known antibiotics because of their location and because treatment failures occur often requiring additional courses of therapy. A particular problem is the increasing use of antibacterial agents and the subsequent resistance of these organisms, e.g., methicillin-resistant Staphylococcus aureus (MRSA), vancomycin resistant Enterococci (VRE), glycopeptide-intermediate Staphylococcus aureus (GISA) and vancomycin resistant Staphylococcus aureus (VISA). (Tenbouris et al., “Methicillin-resistant Staphylococcus aureus: an increasing problem in a diabetic foot clinic,” Diabetic Med. 16:767-771 (1999)).

Drugs proposed for treating diabetic foot include flucloxacillin, cefalexin, metronidazole, amoxicillin and clavulanic acid, clindamycin, ciprofloxacin, fusidic acid, and rifampicin (Avery's Drug Treatment, 4th ed. (1997), p. 74²). Most of these antibiotics proposed for treating diabetic foot are to be taken orally (PO) or intravenously (IV) (Merck Manual p. 1103 1120; Avery's Drug Treatment, 4th ed. (1997), p. 1461-1469) and antibacterial agents are generally administered orally or parenterally due to the low permeability of the antibiotic agents. In addition to the problems noted above, however, adverse side effects sometimes occur from orally-administered antibiotics, including nausea. In addition, due to metabolism by the intended recipient, oral and intravenous doses must be higher than the therapeutically-effective amount to obtain systemic levels in the circulatory system of the mammal to be treated. Likewise, to be effective against non-systemic infections such as diabetic foot infections, the orally or parenterally administered antibiotics must be transported to the site of infection. In diabetic foot infections, circulation is poor so infections may not be able to be treated systemically, and the bacterial infection may result in the need to amputate the foot. Because of this, antibacterial agents are sometimes applied topically for non-systemic infections near the surface of the skin or immediately adjacent to an open wound, although, diabetic foot infections are not “topical infections.” “Topical infections” are known in the art as superficial infections, such as a simple cut.

The following publications, which are hereby incorporated by reference in their entirety, disclose various oxazolidinone antibiotics and methods for producing oxazolidinone antibiotics which are well known to those skilled in the art to have good activity against gram-positive organisms: U.S. Pat. Nos. 6,313,307; 6,239,152; 6,166,056; 6,069,160; 6,051,716; 6,043,266; 5,968,962; 5,952,324; 5,827,857; 5,792,765; 5,698,574; 5,688,792; 5,684,023; 5,652,238; 5,627,181; 5,565,571; 5,547,950; 5,529,998; 5,523,403; 5,254,577; 5,247,090; 5,231,188; 5,225,565 5,182,403 and 5,164,510; 5,043,443; and 4,705,799 and PCT Application 20, and publications PCT/US93/04850, WO94/01110; PCT/US94/08904, WO95/07271; PCT/US95/02972, WO95/25106; PCT/US95/10992, WO96/13502; PCT/US96/05202, WO96/35691; PCT/US96/12766; PCT/US96/13726; PCT/US96/14135; PCT/US96/17120; PCT/US96/19149; PCT/US97/01970; PCT/US95/12751, WO96/15130, PCT/US96/00718, WO96/23788, WO98/54161, WO99/29688, WO97/30995, WO97/09328, WO95/07271, WO00/21960, WO01/40236, WO99/64417, and WO01/81350. These publications disclose various oxazolidinone antibiotics effective against a number of human and veterinary pathogens which can be administered orally, parenterally or topically for the treatment of systemic bacterial diseases in mammals. Linezolid, (S)-N-[[3-[3-fluoro-4-(4-morpholinyl)phenyl]-2-oxo-5-oxazolidinyllmethyl]acetamide, (ZYVOXO, Pharmacia-Upjohn) is an example of a synthetic oxazolidinone antibiotic agent that is active against almost all aerobic gram positive bacteria, including streptococci, MRSA and VRE, as well as certain gram-negative bacteria e.g. Pasteurella multocida and anaerobic bacteria. Linezolid is approved for marketing in the United States, comes in an intravenous preparation, and is highly bioavailable when taken orally. (Stevens, et al., “Randomized Comparison of Linezolid (PNU-100766) Versus Oxocillindicloxacillin for Treatment of Complicated Skin and Soft tissue Infections,” Antimicrob. Agents Chemother. 44: 3408-3414 (2000); Stevens et al., “Linezolid Versus Vancomycin for the Treatment of Methicillin-Resistant Staphylococcus aureus (MRSA) Infections,” Clin. Infec. Dis. 34:1481-1490 (2000); and Zurenko et al., “In Vitro Activities of U-100592 and U-100766, Novel Oxazolidinone Antibacterial Agents,” Antimicrob. Agents Chemother. 40: 839-845 (1996)).

There is a need for systemic pharmaceutical compositions and methods for treating diabetic foot infections which would provide antibacterial agents at therapeutically-effective levels at the site of a diabetic foot infection. In view of the importance of aerobic gram-positive cocci in diabetic foot infections, and the increasing incidence of resistance of these organisms to currently used antibiotics, there is also a need for pharmaceutical compositions and methods for treating diabetic foot infections caused by resistant strains of infectious agents.

### SUMMARY OF THE INVENTION

One aspect of the present invention relates to a method of treating diabetic foot infections in a mammal who is in need of such treatment which comprises parenteral, intravenous, or oral administration of a pharmaceutical formulation containing a pharmaceutically effective amount of an oxazolidinone that treats the infections. The method can include the administration of antibacterially effective amounts of an oxazolidinone in combination with other antibacterial agents.

Another aspect of the present invention relates to the use of a composition comprising a pharmaceutically effective amount of an oxazolidinone for the manufacture of a medicament for preventing or treating a diabetic foot infection.

These and other aspects, advantages, and features of the invention will become apparent from the following detailed description.

### BRIEF DESCRIPTION OF THE DRAWINGS

FIG. 1 is a graph that depicts the overall clinical cure rates in the two populations of patients with diabetic foot who were treated by oral and/or intravenous administration of linezolid, ampicillin/sulbactam, or amoxicillin/clavulanate.

FIG. 2 is a graph that depicts the clinical outcome by primary infection-type diagnosis of patients with diabetic foot who were treated by oral and/or intravenous administration of linezolid, ampicillin/sulbactam, or amoxicillin/clavulanate.

### DETAILED DESCRIPTION OF THE INVENTION

The present invention relates to, the oral, parenteral, or intravenous administration of pharmaceutically effective amounts of an oxazolidinone useful for treating diabetic foot infections. The oral, parenteral, or intravenous activity of an oxazolidinone, for example, linezolid, provides a surprisingly effective activity in treating a non-systemic infection such as a diabetic foot infection.

The following definitions are given for clarification only and are not intended to limit the scope of the application. Dorland's Illustrated Medical Dictionary (29^{th} edition, 2000, p. 1273) defines oral as “pertaining to the mouth, taken through or applied in the mouth, as an oral medication. Therefore, oral administration is administration in-or through the mouth. Dorland's Illustrated Medical Dictionary (29^{th} edition, 2000, p. 1324) defines parenteral as “not through the alimentary canal but rather by injection through some other route, such as subcutaneous, intramuscular, intraorbital, intracapsular, intraspinal, intrastemal, or intravenous.” Therefore, parenteral administrations may include injections to generate a systemic effect or injections directly to the afflicted area, examples of which are subcutaneous, intravenous, intramuscular, intradermal, intrathecal, intraocular, intravetricular, intraorbital, intracapsular, intraspinal, intrastemal, and general infusion techniques.

In addition, Dorland's Illustrated Medical Dictionary (29^{th} edition, 2000, p. 913) defines intravenous as “within a vein or veins.” Therefore, intravenous administration is administration to a vein. Soft tissue describes the extraskeletal connective tissue that accounts for more than 50 percent of body weight and includes muscle, tendon, fat, fascia, and synovium (Oxford Textbook of Surgery, Morris, Peter J. and Malt, Ronald A., eds, (1994), p. 1495. Fascia is defined as a sheet or band of fibrous tissue such as lies deep to the skin or forms an investment for muscles and various organs of the body (Dorland's Illustrated Medical Dictionary 29^{th} edition, 2000, p. 652-654). There are many types of fasciae. Cellulitis is a infection of a type of fascia. Cellulitis is a diffuse inflammation of the soft or connective tissue due to infection, in which a thin, watery exudates spreads through the cleavage places of interstitial and tissue spaces; it may lead to ulceration and absecess (Dorland's Illustrated Medical Dictionary 29^{th} edition, 2000, p. 317). Subcutaneous means beneath the skin (Dorland's Illustrated Medical Dictionary 29^{th} edition, 2000, p. 1718). Synovia is a transparent alkaline viscid fluid, resembling the white of an egg, secreted by the synovial membrane, and contained in joint cavities, bursae, and tendon sheaths (Dorland's Illustrated Medical Dictionary 29^{th} edition, 2000, p. 1773). Bursae are sac or sac-like cavities filled with a viscid fluid and situated at places in the tissues at which friction would otherwise occur (Dorland's Illustrated Medical Dictionary 29 edition, 2000, p. 254). An abscess is a localized collection of pus in a cavity formed by the disintegration of tissues (I)orland's Illustrated Medical Dictionary 29^{th} edition, 2000, p.5-6). An acute abscess in an abscess which runs a relatively short course, introducing some fever and a painful local inflammation. (Dorland's Illustrated Medical Dictionary 29^{th} edition, 2000, p. 6). An abscess is below the surface of the skin.

A method of treating a diabetic foot infection in a mammal who is in need of such treatment comprises parenteral, intravenous, or oral administration of a parenterally, intravenously or orally-effective amount, respectively, of an oxazolidinone. The terms “parenterally-effective amount,” “intravenously-effective amount,” and “orally-effective amount,” as used herein, refer to an amount effective to prevent development of, or to alleviate any existing symptoms of, a diabetic foot infection caused by bacteria.

Useful mammals which are within the scope of the present invention include humans, companion animals such as dogs and cats, or commercially important livestock animals such as horses, cattle and pigs. It is preferred that the mammal be a human, dog or cat; more preferably a human.

Oxazolidinones suitable for the invention typically are gram-positive antibacterial agents. The terms “gram-positive antibiotic” and “gram-positive antibacterial agent” refer to an antibacterial agent active against gram-positive bacterial organisms. The terms “gram-negative antibiotic” and “gram-negative antibacterial agent” refer to an antibacterial agent active against gram-negative bacterial organisms. Certain oxazolidinone compounds and methods for producing oxazolidinone compounds useful in the invention have been described in U.S. Pat. Nos. 6,313,307; 6,239,152; 6,166,056; 6,069,160; 6,051,716; 6,043,266; 5,968,962; 5,952,324; 5,827,857; 5,792,765; 5,698,574; 5,688,792; 5,684,023; 5,652,238; 5,627,181; 5,565,571; 5,547,950; 5,529,998; 5,523,403; 5,254,577; 5,247,090; 5,231,188; 5,225,565; 5,182,403 and 5,164,510; 5,043,443; and 4,705,799 and PCT Application and publications PCT/US93/04850, WO94/01110; PCT/US94/08904, WO95/07271; PCT/US95/02972, WO95/25106; PCT/US95/10992, WO96/13502; PCT/US96/05202, WO96/35691; PCT/US96/12766; PCT/US96/13726; PCT/US96/14135; PCT/US96/17120; PCT/US96/19149; PCT/US97/01970; PCT/US95/12751, WO96/15130, PCT/US96/00718, WO96/23788, WO98/54161, WO99/29688, WO97/30995, WO97/09328, WO95/07271, WO00/21960, WO01/40236, WO99/64417, and WO01/81350, the entire disclosures of which are hereby incorporated by reference in their entirety. Suitable compounds have the formula I:

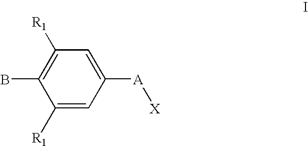

or a pharmaceutically acceptable salt thereof wherein:

A is a structure i, ii, iii, or iv

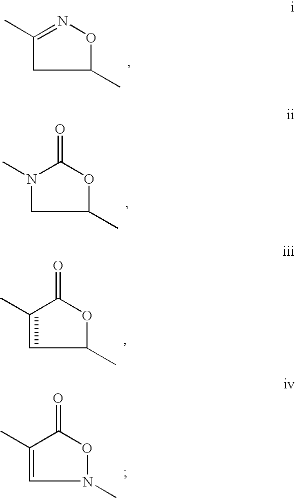

B is selected from cycloalkyl, substituted cycloalkyl, cycloalkenyl, substituted cycloalkenyl, aryl, substituted aryl, het and substituted het, or

B and one R₁ together, with the phenyl carbon atoms to which B and the one R₁ are bonded, form a het, the het optionally being a substituted het;

X is a group selected from —CH₂—NH—C(O)—R₂, —CH₂—R₂, and —CH₂—Y—R₂;

Y is O, S, or —NH—;

R₁ is independently selected from H, alkyl, alkoxy, amino, NO₂, CN, halo, substituted alkyl, substituted alkoxy, and substituted amino; and

R₂ is independently selected from H, —OH, amino, alkyl, substituted alkyl, alkoxy, substituted alkoxy, alkenyl, substituted alkenyl, cycloalkyl, substituted cycloalkyl, cycloalkenyl, substituted cycloalkenyl, het, substituted het, aryl, and substituted aryl.

The following definitions are used, unless otherwise described.

The carbon content of various hydrocarbon-containing moieties is indicated by a prefix designating the minimum and maximum number of carbon atoms in the moiety, i.e., the prefix Cᵢ-Cⱼ defines the number of carbon atoms present from the integer “i” to the integer “j” inclusive. Thus, C₁-C₄ alkyl refers to an alkyl group of 1 to 4 carbon atoms, inclusive, for example, methyl, ethyl, propyl, isopropyl, butyl, and tert-butyl. C₁-C₈ alkyl is methyl, ethyl, propyl, butyl, pentyl, hexyl, heptyl, octyl, and isomeric forms thereof.

The term “halo” refers to a halogen atom selected from Cl, Br, I, and F.

The term “alkyl” refers to both straight- and branched-chain moieties. Unless otherwise specifically stated alkyl moieties include between 1 and 6 carbon atoms.

The term “alkenyl” refers to both straight- and branched-chain moieties containing at least one —C═C—. Unless otherwise specifically stated alkenyl moieties include between 1 and 6 carbon atoms.

The term “alkynyl” refers to both straight- and branched-chain moieties containing at least one —C≡C—. Unless otherwise specifically stated alkynyl moieties include between 1 and 6 carbon atoms.

The term “alkoxy” refers to —O-alkyl groups.

The term “cycloalkyl” refers to a cyclic alkyl moiety. Unless otherwise specifically stated cycloalkyl moieties will include between 3 and 9 carbon atoms.

The term “cycloalkenyl” refers to a cyclic alkenyl moiety. Unless otherwise specifically stated cycloalkyl moieties will include between 3 and 9 carbon atoms and at least one —C═C— group within the cyclic ring.

The term “amino” refers to —NH_{2.}

The term “aryl” refers to phenyl, phenyl, and naphthyl.

The term “het” refers to mono- or bi-cyclic ring systems containing at least one heteroatom selected from O, S, and N. Each mono-cyclic ring may be aromatic, saturated, or partially unsaturated. A bi-cyclic ring system may include a mono-cyclic ring containing at least one heteroatom fused with a cycloalkyl or aryl group. A bi-cyclic ring system may also include a mono-cyclic ring containing at least one heteroatom fused with another het, mono-cyclic ring system.

Examples of “het” include, but are not limited to, pyridine, thiophene, furan, pyrazoline, pyrimidine, 2-pyridyl, 3-pyridyl, 4-pyridyl, 2-pyrimidinyl, 4-pyrimidinyl, 5-pyrimidinyl, 3-pyridazinyl, 4-pyridazinyl, 3-pyrazinyl, 4-oxo-2-imidazolyl, 2-imidazolyl, 4-imidazolyl, 3-isoxazolyl, 4-isoxazolyl, 5-isoxazolyl, 3-pyrazolyl, 4-pyrazolyl, 5-pyrazolyl, 2-oxazolyl, 4-oxazolyl, 4-oxo-2-oxazolyl, 5-oxazolyl, 1,2,3-oxathiazole, 1,2,3-oxadiazole, 1,2,4-oxadiazole, 1,2,5-oxadiazole, 1,3,4-oxadiazole, 2-thiazolyl, 4-thiazolyl, 5-thiazolyl, 3-isothiazole, 4-isothiazole, 5-isothiazole, 2-furanyl, 3-furanyl, 2-thienyl, 3-thienyl, 2-pyrrolyl, 3-pyrrolyl, 3-isopyrrolyl, 4-isopyrrolyl, 5-isopyrrolyl, 1,2,3,-oxathiazole-1-oxide, 1,2,4-oxadiazol-3-yl, 1,2,4-oxadiazol-5-yl, 5-oxo-1,2,4-oxadiazol-3-yl, 1,2,4-thiadiazol-3-yl, 1,2,4-thiadiazol-5-yl, 3-oxo-1,2,4-thiadiazol-5-yl, 1,3,4-thiadiazol-5-yl, 2-oxo-1,3,4-thiadiazol-5-yl, 1,2,4-triazol-3-yl, 1,2,4-triazol-5-yl, 1,2,3,4-tetrazol-5-yl, 5-oxazolyl, 3-isothiazolyl, 4-isothiazolyl, 5-isothiazolyl, 1,3,4,-oxadiazole, 4-oxo-2-thiazolinyl, 5-methyl-1,3,4-thiadiazol-2-yl, thiazoledione, 1,2,3,4-thiatriazole, 1,2,4-dithiazolone, phthalimide, quinolinyl, morpholinyl, benzoxazoyl, diazinyl, triazinyl, quinolinyl, quinoxalinyl, naphthyridinyl, azetidinyl, pyrrolidinyl, hydantoinyl, oxathiolanyl, dioxolanyl, imidazolidinyl, piperazinyl, thiopyranyl, oxazolidinyl, thiophenyl, thiomorpholino, and azabicyclo[2.2.1]heptyl.

The term “substituted alkyl” refers to an alkyl moiety including 1-4 substituents selected from halo, het, cycloalkyl, cycloalkenyl, aryl, —OQ₁₀, —SQ₁₀, —S(O)₂Q₁₀, —S(O)Q₁₀, —OS(O)₂Q₁₀, —C(═NQ₁₀)Q₁₀, —SC(O)Q₁₀, —NQ₁₀Q₁₀, —C(O)Q₁₀, —C(S)Q₁₀, —C(O)OQ₁₀, —OC(O)Q₁₀, —C(O)NQ₁₀Q₁₀, —C(O)C(Q₁₆)₂OC(O)Q₁₀, —CN, ═O, ═S, —NQ₁₀C(O)Q₁₀, —NQ₁₀C(O)NQ₁₀Q₁₀, —S(O)₂NQ₁₀Q₁₀, —NQ₁₀S(O)₂Q₁₀, —NQ₁₀S(O)Q₁₀, —NQ₁₀SQ₁₀, —NO₂, and —SNQ₁₀Q₁₀. Each of the het, cycloalkyl, cycloalkenyl, and aryl being optionally substituted with 1-4 substituents independently selected from halo and Q₁₅.

The term “substituted aryl” refers to an aryl moiety having 1-3 substituents selected from —OQ₁₀, —SQ₁₀, —S(O)₂Q₁₀, —S(O)Q₁₀, —OS(O)₂Q₁₀, —C(═NQ₁₀)Q₁₀, —SC(O)Q₁₀, —NQ₁₀Q₁₀, —C(O)Q₁₀, —C(S)Q₁₀, —C(O)OQ₁₀, —OC(O)Q₁₀, —C(O)NQ₁₆Q₁₀, —C(O)C(Q₁₆)₂OC(O)Q₁₀, —CN, ═O, ═S, —NQ₁₀C(O)Q₁₀, —NQ₁₀C(O)NQ₁₀Q₁₀, —S(O)₂NQ₁₀Q₁₀, —NQ₁₀S(O)₂Q₁₀, —NQ₁₀S(O)Q₁₀, —NQ₁₀SQ₁₀, —NO₂, —SNQ₁₀Q₁₀, alkyl, substituted alkyl, het, halo, cycloalkyl, cycloalkenyl, and aryl. The het, cycloalkyl, cycloalkenyl, and aryl being optionally substituted with 1-3 substituents selected from halo and Q₁₅.

The term “substituted het” refers to a het moiety including 1-4 substituents selected from —OQ₁₀, —SQ₁₀, —S(O)₂Q₁₀, —S(O)Q₁₀, —OS(O)₂Q₁₀, —C(═NQ₁₀)Q₁₀, —SC(O)Q₁₀, —NQ₁₀Q₁₀, —C(O)Q₁₀, —C(S)Q₁₀, —C(O)OQ₁₀, —OC(O)Q₁₀, —C(O)NQ₁₀Q₁₀, —C(O)C(Q₁₆)₂OC(O)Q₁₀, —CN, ═O, ═S, —NQ₁₀C(O)Q₁₀, —NQ₁₀C(O)NQ₁₀Q₁₀, —S(O)₂NQ₁₀Q₁₀, —NQ₁₀S(O)₂Q₁₀, —NQ₁₀S(O)Q₁₀, —NQ₁₀SQ₁₀, —NO₂, —SNQ₁₀Q₁₀, alkyl, substituted alkyl, het, halo, cycloalkyl, cycloalkenyl, and aryl. The het, cycloalkyl, cycloalkenyl, and aryl being optionally substituted with 1-3 substituents selected from halo and Q₁₅.

The term “substituted alkenyl” refers to a alkenyl moiety including 1-3 substituents —OQ₁₃, —SQ₁₀, —S(O)₂Q₁₀, —S(O)Q₁₀, —OS(O)₂Q₁₀, —C(═NQ₁₀)Q₁₀, —SC(O)Q₁₀, —NQ₁₀Q₁₀, —C(O)Q₁₀, —C(S)Q₁₀, —C(O)OQ₁₀, —OC(O)Q₁₀, —C(O)NQ₁₀Q₁₀, —C(O)C(Q₁₆)₂OC(O)Q₁₀, —CN, ═O, ═S, —NQ₁₀C(O)Q₁₀, —NQ₁₀C(O)NQ₁₀Q₁₀, —S(O)₂NQ₁₀Q₁₀, —NQ₁₀S(O)₂Q₁₀, —NQ₁₀S(O)Q₁₀, —NQ₁₀SQ₁₀, —NO₂, —SNQ₁₀Q₁₀, alkyl, substituted alkyl, het, halo, cycloalkyl, cycloalkenyl, and aryl. The het, cycloalkyl, cycloalkenyl, and aryl being optionally substituted with 1-3 substituents selected from halo and Q₁₅.

The term “substituted alkoxy” refers to an alkoxy moiety including 1-3 substituents —OQ₁₀, —SQ₁₀, —S(O)₂Q₁₀, —S(O)Q₁₀, —OS(O)₂Q₁₀, —C(═NQ₁₀)Q₁₀, —SC(O)Q₁₀, —NQ₁₀Q₁₀, —C(O)Q₁₀, —C(S)Q₁₀, —C(O)OQ₁₀, —OC(O)Q₁₀, —C(O)NQ₁₀Q₁₀, —C(O)C(Q₁₆)₂OC(O)Q₁₀, —CN, ═O, ═S, —NQ₁₀C(O)Q₁₀, —NQ₁₀C(O)NQ₁₀Q₁₀, —S(O)₂NQ₁₀Q₁₀, —NQ₁₀S(O)₂Q₁₀, —NQ₁₀S(O)Q₁₀, —NQ₁₀SQ₁₀, —NO₂, —SNQ₁₀Q₁₀, alkyl, substituted alkyl, het, halo, cycloalkyl, cycloalkenyl, and aryl. The het, cycloalkyl, cycloalkenyl, and aryl being optionally substituted with 1-3 substituents selected from halo and Q₁₅.

The term “substituted cycloalkenyl” refers to a cycloalkenyl moiety including 1-3 substituents —OQ₁₀, —SQ₁₀, —S(O)₂Q₁₀, —S(O)Q₁₀, —OS(O)₂Q₁₀, —C(═NQ₁₀)Q₁₀, —SC(O)Q₁₀, —NQ₁₀Q₁₀, —C(O)Q₁₀, —C(S)Q₁₀, —C(O)OQ₁₀, —OC(O)Q₁₀, —C(O)NQ₁₀Q₁₀, —C(O)C(Q₁₆)₂OC(O)Q₁₀, —CN, ═O, ═S, —NQ₁₀C(O)Q₁₀, —NQ₁₀C(O)NQ₁₀Q₁₀, —S(O)₂NQ₁₀Q₁₀, —NQ₁₀S(O)₂Q₁₀, —NQ₁₀S(O)Q₁₀, —NQ₁₀SQ₁₀, —NO₂, —SNQ₁₀Q₁₀, alkyl, substituted alkyl, het, halo, cycloalkyl, cycloalkenyl, and aryl. The het, cycloalkyl, cycloalkenyl, and aryl being optionally substituted with 1-3 substituents selected from halo and Q₁₅.

The term “substituted amino” refers to an amino moiety in which one or both of the amino hydrogens are replaced with a group selected from —OQ₁₀, —SQ₁₀, —S(O)₂Q₁₀, —S(O)Q₁₀, —OS(O)₂Q₁₀, —C(═NQ₁₀)Q₁₀, —SC(O)Q₁₀, —NQ₁₀Q₁₀, —C(O)Q₁₀, —C(S)Q₁₀, —C(O)OQ₁₀, —OC(O)Q₁₀, —C(O)NQ₁₀Q₁₀, —C(O)C(Q₁₆)₂OC(O)Q₁₀, —CN, ═O, ═S, —NQ₁₀C(O)Q₁₀, —NQ₁₀C(O)NQ₁₀Q₁₀, —S(O)₂NQ₁₀Q₁₀, —NQ₁₀S(O)₂Q₁₀, —NQ₁₀S(O)Q₁₀, —NQ₁₀SQ₁₀, —NO₂, —SNQ₁₀Q₁₀, alkyl, substituted alkyl, het, halo, cycloalkyl, cycloalkenyl, and aryl. The het, cycloalkyl, cycloalkenyl, and aryl being optionally substituted with 1-3 substituents selected from halo and Q₁₅.

Each Q₁₀ is independently selected from —H, alkyl, cycloalkyl, het, cycloalkenyl, and aryl. The het, cycloalkyl, cycloalkenyl, and aryl being optionally substituted with 1-3 substituents selected from halo and Q₁₃.

Each Q₁₁, is independently selected from —H, halo, alkyl, aryl, cycloalkyl, and het. The alkyl, aryl, cycloalkyl, and het being optionally substituted with 1-3 substituents independently selected from halo, —NO₂, —CN, ═S, ═O, and Q]₄.

Each Q₁₃ is independently selected from Q₁₁, —OQ₁₁, —SQ₁₁, —S(O)₂Q₁₁, —S(O)Q₁₁, —OS(O)₂Q₁₁, —C(═NQ₁₁)Q₁₁, —SC(O)Q₁₁, —NQ₁₁Q₁₁, —C(O)Q₁₁, —C(S)Q₁₁, —C(O)OQ₁₁, —OC(O)Q₁₁, —C(O)NQ₁₁Q₁₁, —C(O)C(Q₁₆)₂OC(O)Q₁₀, —CN, ═O, ═S, —NQ, C(O)Q₁₁, —NQ₁₁C(O)NQ₁₁Q₁₁, —S(O)₂NQ₁₁Q₁₁, —NQ, S(O)₂Q₁₁, —NQ₁₁S(O)Q₁₁, —NQ₁₁SQ₁₁, —NO₂, and —SNQ₁₁Q₁₁.

Each Q₁₄ is —H or a substituent selected from alkyl, cycloalkyl, cycloalkenyl, phenyl, or naphthyl, each optionally substituted with 1-4 substituents independently selected from —F, —Cl, —Br, —I, —OQ₁₆, —SQ₁₆, —S(O)₂Q₁₆, —S(O)Q₁₆, —OS(O)₂Q₁₆, —NQ₁₆Q₁₆, —C(O)Q₁₆, —C(S)Q₁₆, —C(O)OQ₁₆, —NO₂, —C(O)NQ₁₆Q₁₆, —CN, —NQ₁₆C(O)Q₁₆, —NQ₁₆C(O)NQ₁₆Q₁₆, —S(O)₂NQ₁₆Q₁₆, and —NQ₁₆S(O)₂Q₁₆. The alkyl, cycloalkyl, and cycloalkenyl being further optionally substituted with ═O or ═S.

Each Q₁₅ is alkyl, cycloalkyl, cycloalkenyl, het, phenyl, or naphthyl, each optionally substituted with 1-4 substituents independently selected from —F, —Cl, —Br, —I, —OQ₁₆, —Q₁₆, —S(O)₂Q₁₆, —S(O)Q₁₆, —OS(O)₂Q₁₆, —C(═NQ₁₆)Q₁₆, —SC(O)Q₁₆, —NQ₁₆Q₁₆, —C(O)Q₁₆, —C(S)Q₁₆, —C(O)OQ₁₆, —OC(O)Q₁₆, —C(O)NQ₁₆Q₁₆, —C(O)C(Q₁₆)₂OC(O)Q₁₆, —CN, —NQ₁₆C(O)Q₁₆, —NQ₁₆C(O)NQ₁₆Q₁₆, —S(O)₂NQ₁₆Q₁₆, —NQ₁₆S(O)₂Q₁₆, —NQ₁₆S(O)Q₁₆, —NQ₁₆SQ₁₆, —NO₂, and —SNQ₁₆Q₁₆. The alkyl, cycloalkyl, and cycloalkenyl being further optionally substituted with ═O or ═S.

Each Q₁₆ is independently selected from —H, alkyl, and cycloalkyl. The alkyl and cycloalkyl optionally including 1-3 halos.

In certain embodiments, the oxazolidinone can have the formulas II or III:

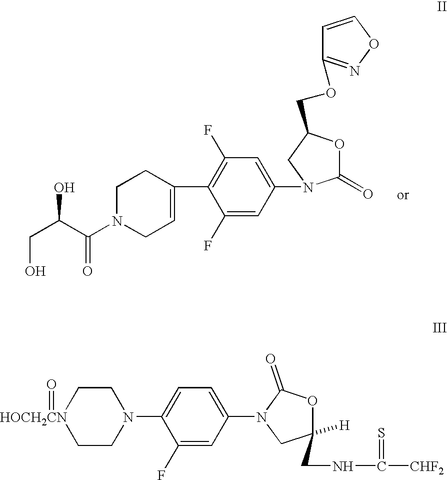

Oxazolidinones suitable for the invention typically are gram-positive antibacterial agents. Certain oxazolidinone compounds useful in the invention have been described in U.S. Pat. No. 5,688,792, the entire disclosure of which is incorporated herein by reference. Other suitable oxazolidinone compounds have the following formula IV:

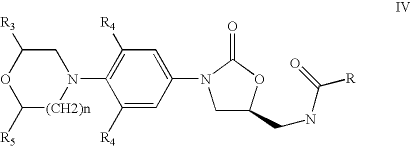

or is a pharmaceutically acceptable salt thereof, wherein:

n is 0, 1, or 2;

R is selected from the group consisting of:

hydrogen;

C₁-C₈ alkyl optionally substituted with one or more substituents selected from the group consisting of F, Cl, hydroxy, C₁-C₈ alkoxy, C₁-C₈ acyloxy, or —CH₂-phenyl;

C₃-C₆ cycloalkyl;

amino;

C₁-C₈ alkylamino;

C₁-C₈ dialkylamino; or

C₁-C₈ alkoxy;

R³ at each occurrence is independently selected from the group consisting of H, CH₃, CN, CO₂H, CO₂R, and (CH₂)ₘR⁶, wherein m is 1 or 2;

R⁴ at each occurrence is independently selected from the group consisting of H, F, and Cl;

R⁵ is H or CH₃;

R⁶ is selected from the group consisting of H, OH, OR, OCOR, NH₂, NHCOR, and N(R⁷)₂; and

R⁷ at each occurrence is independently selected from the group consisting of H, p-toluensulfonyl, and C₁-C₄ alkyl optionally substituted with one or more substituents selected from the group consisting of Cl, F, OH, C₁-C₈ alkoxy, amino, C₁-C₉ alkylamino, and C₁-C₈ dialkylamino.

Additionally suitable oxazolidinones compounds have the following formula V:

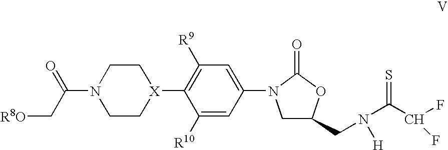

or a pharmaceutically acceptable salt thereof, wherein:

X is N or CH;

R⁹ and R₁₀ are independently H or F; and

R⁸ is H, benzyl, or —C(═O)C₁₋₄alkyl.

Compounds of formula V can be prepared as illustrated in Schemes I and II, wherein X, R⁸, R⁹ and R¹⁰ are as described previously or in claims. In Scheme I, R¹¹ represents hydrogen, —C(═O)CH₂OR⁸ or suitable amine protecting groups such as tert-butoxycarbonyl (Boc) and benzyloxycarbonyl (Cbz). The starting material, amines (VI), can be prepared according to the procedure described in U.S. Pat. No. 6,342,523. Where R¹¹ in the amine VI is —C(═O)CH₂OR⁸ or suitable amine protecting groups, they are allowed to react with an ester of difluoroethanethioic O-acid VIII wherein R¹² is C₁₋₄ alkyl optionally substituted by one or two phenyl groups. Suitable solvents for this reaction include methanol, chloroform, methylene chloride or mixtures thereof at temperatures of about 10° C. to about 30° C. A tertiary amine base such as triethylamine can be used to facilitate this reaction, especially if a salt of the amine VI is employed. The Boc protecting group can be removed with acid catalysts such as trifluoroacetic acid in methylene chloride or 4N hydrogen chloride in dioxane at temperatures of about 0° C. to about 25° C. Removal of the Cbz group can be carried out with about 20% hydrogen bromide in acetic acid at temperatures about 0° C. to about 30° C. The remaining steps which lead from the resulting compounds wherein R¹¹ is hydrogen to compounds V are shown in Scheme II.

An alternative method for preparing compounds of formula V is illustrated in Scheme II. Condensation of a compound of structure IX, wherein R¹³ is a protecting group such as Boc or Cbz, with difluoroacetic acid provides the difluoroacetamide X. Reagents and conditions for this condensation include the use of 1-(3-dimethylaminopropyl)-3-ethylcarbodiimide hydrochloride, (EDC) with 4-(dimethylamino)pyridine (DMAP) in pyridine at temperature of about 0° C. to about 25° C. or EDC with 1-hydroxybenzotriazole hydrate (HOBT) and triethylamine in DMF at temperature of about 0° C. to about 25° C. The protecting groups R¹³ can then be removed to give compounds XI which can be converted to the thioamide XII with Lawesson's Reagent. The reaction of XI with Lawesson's Reagent is facilitated by the use of 1,3-dimethyl-3,4,5,6-tetrahydro-2(1H)-pyrimidone (DMPU) and can be carried out in solvents such as THF or dioxane at temperatures of about 20° C. to about 100° C. Condensation of the amines XII with activated carboxylic acid derivatives will then give compounds of formula V. The reaction of XII with acetoxyacetyl chloride and triethylamine in methylene chloride at temperature of about 0° C. to about 25° C., for example, can be used to prepare compounds V where R⁸ is acetyl. Condensing agents such as EDC with the appropriate acids, as described above, can also be used for this reaction. Compounds where R⁸ is acetyl can be hydrolyzed to the corresponding compounds where R⁸ is hydrogen with aqueous potassium carbonate in methanol.

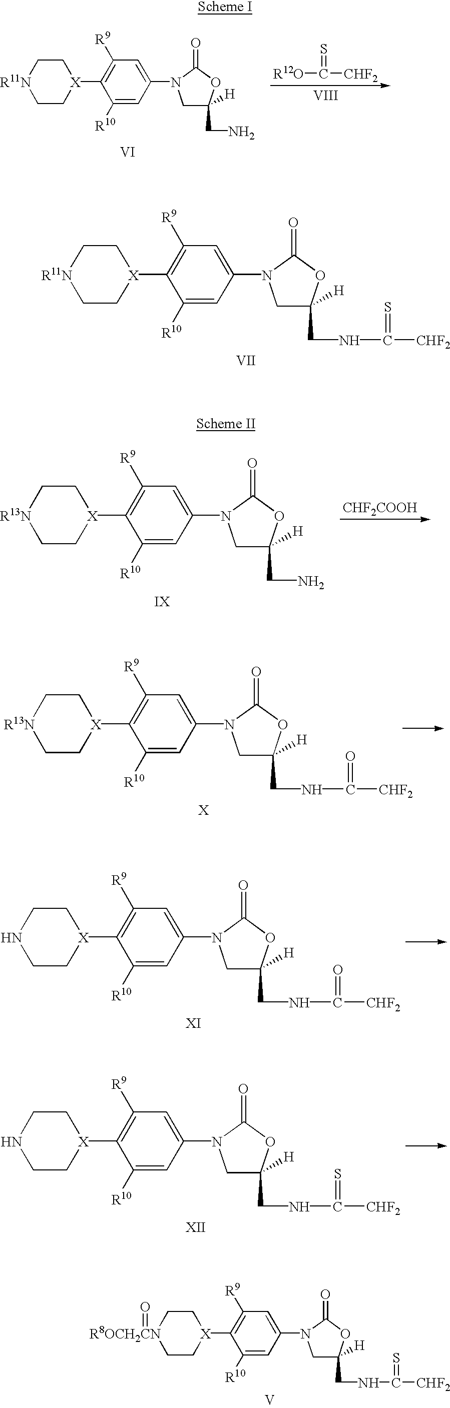

As used herein, the term “pharmaceutically acceptable salts” refers to organic and inorganic acid addition salts of the parent compound. Examples of pharmaceutically acceptable salts are organic acid addition salts formed with acids which form a physiologically acceptable anion, for example, tosylate, methanesulfonate, acetate, citrate, malonate, tartarate, succinate, benzoate, ascorbate, etoglutarate, and glycerophosphate. Suitable inorganic salts may also be formed, including hydrochloride, hydrobromide, hydroiodide, sulfate, phosphate, acetate, propionate, lactate, mesylate, maleate, mal ate, succinate, tartrate, citrate, 2-hydroxyethyl sulfate, fumarate, nitrate, bicarbonate, carbonate, and the like.

Pharmaceutically acceptable salts may be obtained using standard procedures well known in the art, for example, reacting a sufficiently basic compound such as an amine with a suitable acid affording a physiologically acceptable anion. Alkali metal (for example, sodium, potassium or lithium) or alkaline earth metal (for example calcium) salts of carboxylic acids can also be made.

One suitable oxazolidinone compound having the structure,

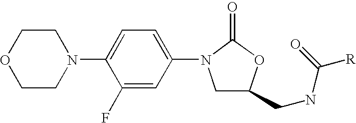

has the IUPAC name (S)-N-[[3-[3-fluoro-4-(4-morpholinyl)phenyl]-2-oxo-5-oxazolidinyl]methyl]acetamide. The compound is commonly known as linezolid and has demonstrated particularly effective anti-bacterial activity.

The linezolid compound can be prepared according to any suitable method, including for example, general methods described in U.S. Pat. No. 5,688,792, the entire disclosure of which is herein incorporated by reference. Briefly, the heteroaryl substituent, for example an oxazine or thiazine moiety, is reacted with a functionalized nitrobenzene in the presence of a suitable base, preferably in an organic solvent, such as acetonitrile, tetrahydrofuran, or ethyl acetate. The nitro group is reduced either by hydrogenation or using a suitable reducing agent, for, example aqueous sodium hydrosulfite, to afford an anilo compound. The anilo compound is converted into its benzyl or methyl urethane derivative, deprotonated with a lithium reagent to give a suitable lithiated intermediate, and treated with (−)-(R)-glycidyl butyrate to afford a crude oxazolidinone compound. A suitable method for preparing the linezolid compound is more particularly described in Example 5 of U.S. Pat. No. 5,688,792. Linezolid can exist in at least two crystal forms as disclosed in U.S. Ser. No. 09/886,641.

Another suitable compound having the structure,

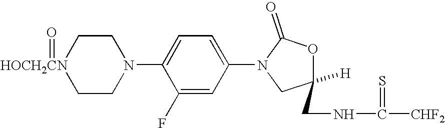

has the IUPAC name 2,2-difluoro-N-({(5S)-3-[3-fluoro-4-(4-glycoloylpiperazin-1-yl)phenyl]-2-oxo-1,3-oxazolidin-5-yl}methyl)ethanethioamide.

According to the invention, an oxazolidinone compound having similar structure or physiochemical properties as any oxazolidinone compound described above will be expected to treat diabetic foot infections. To identify such oxazolidinone compounds, the compound to be tested can substitute linezolid, or any compound of the general oxazolidinone structure in the method of the invention and analyzed for activity for treating diabetic foot infections by any suitable method.

The methods of the invention are particularly effective against resistant strains of bacterial infection including, for example, resistant strains of Staphyloccus aureus. More particularly, the methods and compositions of the invention can be useful in treating diseases caused by MRSA, VRE, GISA, or VISA. The oxazolidinones of the present invention also treat gram-negative infections caused by anaerobes such as Bacteroides fragilis. The oxazolidinone can be combined with other antibiotics to treat infections caused by a broader spectrum of gram-negative and/or gram-positive microorganisms. These infections include skin-associated soft-tissue infections (including subcutaneous tissue infections, abscesses, or myostis) where the gram-positive bacteria are present in the epidermis, dermis, fat layer, and/or muscle layers underlying the epidermis. Likewise, in patients with spreading cellulites or more deep-seated infection, antibiotic therapy becomes necessary. The foot can have sores on it unrelated to the bacterial infection that occur below the surface of the skin of the foot in the soft tissue.

It is apparent to one skilled in the art that a subject is in need of treatment for a diabetic foot infection when the subject has signs and symptoms which may include: purulent or non-purulent drainage or discharge, erythema, fluctuance, heat or localized warmth, pain or tenderness to palpation, an inflamed, reddened, swollen, indurated or tender area on the foot under broken or unbroken skin and which may be coupled with a fever. Soft-tissue infections are treated by administering the desired oxazolidinone orally, paternterally, or intravenously by use of the appropriate pharmaceutical dosage form.

The pharmaceutical compositions and formulations of the present invention can include pharmaceutically acceptable carriers to facilitate the administration of the active agents. As used herein, the term “pharmaceutically acceptable” refers to those properties and/or substances which are acceptable to the patient from a pharmacological/toxicological point of view and to the manufacturing pharmaceutical chemist from a physical/chemical point of view regarding composition, formulation, stability, patient acceptance and bioavailability.

Pharmaceutical compositions of comprising the oxazolidinone antibiotics of the present invention, either individually or in combination with other antibiotics, may be prepared by methods well known in the art, e.g., by means of conventional mixing, dissolving, granulation, dragee-making, levigating, emulsifying, encapsulating, entrapping, lyophilizing processes or spray drying.

Any conventional pharmaceutical preparation can be used. The pharmaceutical composition for use in accordance with the present invention generally will comprise and an effective dose of the active substance and one or more physiologically acceptable carriers comprising excipients and auxiliaries which facilitate processing of the active compounds into preparations which can be used pharmaceutically. Proper formulation is dependent upon the route of administration chosen.

For oral administration, the compounds can be formulated by combining the active compounds with pharmaceutically acceptable carriers well known in the art. Such carriers enable the compounds of the invention to be formulated as tablets, pills, lozenges, powders, dragees, capsules, liquids, solutions, emulsions, gels, syrups, slurries, suspensions, other useful mediums for delivering the active agent, and the like, for oral ingestion by a patient. A carrier can be at least one substance which may also function as a diluent, flavoring agent, solubilizer, lubricant, suspending agent, binder, tablet disintegrating agent, and encapsulating agent. Examples of such carriers or excipients include, but are not limited to, magnesium carbonate, magnesium stearate, talc, sugar, lactose, sucrose, pectin, dextrin, mannitol, sorbitol, starches, gelatin, cellulosic materials, low melting wax, cocoa butter or powder, polymers such as polyethylene glycols, colloidal silica, povidone, and other pharmaceutical acceptable materials.

Dragee cores are provided with suitable coatings. For this purpose, concentrated sugar solutions may be used which may optionally contain gum arabic, talc, polyvinyl pyrrolidone, carbopol gel, polyethylene glycol, and/or titanium dioxide, lacquer solutions, and suitable organic solvents or solvent mixtures. Dyestuffs or pigments may be added to the tablets or dragee coatings for identificatin or to characterize different combinations of active compound doses.

Pharmaceutical compositions which can be used orally include push-fit capsules made of gelatin, as well as soft, sealed capsules made of gelatin and a plasticizer, such as glycerol or sorbitol. The push-fit capsules can contain the active ingredients in admixture with a filler such as lactose, a binder such as starch, and/or a lubricant such as talc or magnesium stearate and, optionally, stabilizers. In soft capsules, the active compounds may be dissolved or suspended in suitable liquids, such as fatty oils, liquid paraffin, liquid polyethylene glycols, cremophor, capmul, medium or long chain mono-, di- or triglycerides. Stabilizers may be added in these formulations, also.

Liquid form compositions include solutions, suspensions and emulsions. For example, there may be provided solutions of the compounds of this invention dissolved in water and water-propylene glycol and water-polyethylene glycol systems, optionally containing suitable conventional coloring agents, flavoring agents, stabilizers and thickening agents.

The compounds may also be formulated for parenteral administration, e.g., by injections, bolus injection or continuous infusion. Formulations for parenteral administration may be presented in unit dosage form, e.g., in ampoules or in multi-dose containers, with an added preservative. The compositions may take such forms as suspensions, solutions or emulsions in oily or aqueous vehicles, and may contain formulating materials such as suspending, stabilizing and/or dispersing agents.

For injection, the compounds of the invention may be formulated in aqueous solution, preferably in physiologically compatible buffers or physiological saline buffer. Suitable buffering agents include trisodium orthophosphate, sodium bicarbonate, sodium citrate, N-methylglucamine, L(+)-lysine and L(+)-arginine.

The compounds or compositions can also be administered intravenously or intraperitoneally by infusion or injection. Solutions of the active compound or its salts can be prepared in water, optionally mixed with a nontoxic surfactant. Dispersions can also be prepared in glycerol, liquid polyethylene glycols, triacetin, and mixtures thereof and in oils. Under ordinary conditions of storage and use, these preparations contain a preservative to prevent the growth of microorganisms.

Pharmaceutical dosage forms suitable for injection or infusion can include sterile aqueous solutions or dispersions or sterile powders comprising the active ingredient which are adapted for the extemporaneous preparation of sterile injectable or infusible solutions or dispersions, optionally encapsulated in liposomes. In all cases, the ultimate dosage form should be sterile, fluid and stable under the conditions of manufacture and storage. The liquid carrier or vehicle can be a solvent or liquid dispersion medium comprising, for example, water, ethanol, a polyol (for example, glycerol, propylene glycol, liquid polyethylene glycols, and the like), vegetable oils, nontoxic glyceryl esters, and suitable mixtures thereof. The proper fluidity can be maintained, for example, by the formation of liposomes, by the maintenance of the required particle size in the case of dispersions or by the use of surfactants. The prevention of the action of microorganisms can be brought about by various antibacterial and antifungal agents, for example, parabens, chlorobutanol, phenol, sorbic acid, thimerosal, and the like. In many cases, it will be preferable to include isotonic agents, for example, sugars, buffers or sodium chloride. Prolonged absorption of the injectable compositions can be brought about by the use in the compositions of agents delaying absorption, for example, aluminum monostearate and gelatin.

Sterile injectable solutions can be prepared by incorporating the active compound in the required amount in the appropriate solvent with various of the other ingredients enumerated above, as required, followed by filter sterilization. In the case of sterile powders for the preparation of sterile injectable solutions, the preferred methods of preparation are vacuum drying and the freeze drying techniques, which yield a powder of the active ingredient plus any additional desired ingredient present in the previously sterile-filtered solutions.

Other parenteral administrations also include aqueous solutions of a water soluble form, such as, without limitation, a salt, of the active compound. Additionally, suspensions of the active compounds may be prepared in a lipophilic vehicle. Suitable lipophilic vehicles include fatty oils such as sesame oil, synthetic fatty acid esters such as ethyl oleate and triglycerides, or materials such as liposomes. Aqueous injection suspensions may contain substances which increase the viscosity of the suspension, such as sodium carboxymethyl cellulose, sorbitol, or dextran. Optionally, the suspension may also contain suitable stabilizers and/or agents that increase the solubility of the compounds to allow for the preparation of highly concentrated solutions.

Alternatively, the active ingredient may be in a powder form for constitution with a suitable vehicle, e.g., sterile, pyrogen-free water, before use.

Additionally, the compounds may be delivered using a sustained-release system. Various sustained-release materials have been established and are well known by those skilled in the art. Sustained-release capsules may, depending on their chemical nature, release the compounds for 24 hours up to several days. Depending on the chemical nature and the biological stability of the therapeutic reagent, additional strategies for protein stabilization may be employed.

An aqueous solution for parenteral or intravenous (“IV”) administration can be placed in a suitable container such as a bag, a bottle, a vial, a large volume parenteral, a small volume parenteral, a syringe, a prefilled syringe or a cassette. As used herein, the term “bottle” to refers to larger bottles, typically having a fill volume, i.e. the amount of liquid contained in an unused product, of at least 20 mL. The term “vials” as used herein refer to smaller bottle-shaped containers, typically having a fill volume of less than 20 mL, for example in units of 1 mL, 2 mL, 5 mL, and the like. It is preferred that the container is bag, a bottle, a vial or a prefilled syringe. For parenteral administration, the more preferred container is a parenteral or syringe. For IV administration, the more preferred container is a bag or bottle, and the most preferred container is a bag. When so used, it is preferred that the bag has sufficient capability to hold 25 mL to 2,000 mL of IV solution. For a bag, amounts of 100 mL, 200 mL, or 300 mL portions of solution are preferred for each bag. However, larger and/or smaller volumes also are acceptable.

The intravenously administered solution is introduced into the patient as a sterile liquid. While there are a number of methods to sterilize an IV solution, it is preferred that the IV solution is sterilized by terminally moist heat or steam sterilization. When the term terminally “moist heat sterilize” is used, it refers to and includes steam sterilization.

To sterilize the solution using terminally moist heat sterilization, the solution is placed in the container suitable for transporting the solution and as a receptacle for holding the solution during administration of the solution. Accordingly, the container is chosen in such a manner as to avoid reacting with the pharmaceutically active ingredient, for example an oxazolidinone compound, during sterilization, transport, or administration.

A container comprising at least 50% polyolefin provides a significant advantage in the storage of linezolid solutions, in particular. One desirable benefit of polyolefin-type containers is that the loss of linezolid during and following terminal moist heat sterilization is minimized. It is particularly beneficial when the primary container-solution contact surface material is the polyolefin. The remainder of the container can be made from polyolefin or other materials. It is preferred that the container-solution contact surface is made from about 50% to about 100% polyolefin. A more preferred container-solution contact surface has from about 70% to about 90% polyolefin. An even more preferred container-solution contact surface comprises from, about 75% to about 85% polyolefin.

Polyolefins include, for example, polyethylenes, polypropylenes, polybutenes, polyisoprenes, and polypentenes and copolymers and mixtures thereof. It is preferred that the polyolefin is polyethylene or polypropylene. A preferred polyolefin is polypropylene or mixture of polypropylene and polyethylene.

Typically, the antibacterial oxazolidinone can be administered 1 to 4 times daily, depending on the place of the infection, the severity of the disease, the size and the age of the patient. In pediatric patients, the adult dose is appropriately reduced for the child based on the size of the child. Oxazolidinones clear very rapidly from the body in young children, particularly those children having less than or about five years of age. Accordingly, a patient of about five years of age or less may require an appropriately adjusted dose three times a day administration. Also, patients who do not respond well to once daily dosing may require four times a day administration. In general, daily administration, until 24 hours after the body temperature returns to normal and/or the redness, swelling and/or inflammation is gone, is preferred.

The amounts of the active agents to be administered can be readily determined by any method available to one with skill in the art of providing therapeutic treatments. To guide the reader in the practice of the invention, generally an amount of from about 200 mg to about 900 mg of the oxazolidinone is administered to the patient, typically either once a day to four times a day. Preferably, the amount of the oxazolidinone is about 500 mg to about 700 mg every 12 hours. A course of treatment for an adult patient can last from about seven days to about 60 days. Other sanitary precautions should be utilized as are known to those skilled in the art

The response of the patient to the treatment can be followed by standard clinical, radiological, microbiological, and other laboratory investigations. In particular, serum cidal assays can be carried out to generate an inhibitor or cidal titer to aid in determining the specific dose to the patient. Typically, the treatment will last from about 7 days to about 28 days. For young-children, especially those about age five and under, the preferred dose is about 10 mg/kg twice daily.

“Treating a diabetic foot infection” in a mammal who is in need of such treatment, means the mammal has a diabetic foot infection which is causing it a problem, including a fever, pain, abscess, or inflammation of a tissue or wound. Treating infections means administering to the mammal oxazolidinone such that the mammal obtains sufficient concentration of the oxazolidinone in the affected area to either kill the existing microorganisms, stop them from growing, and/or reduce their rate of multiplication (increase) to a point where the body's natural defense mechanism can reduce or eradicate the unwanted microorganisms to a level which does not cause clinical problems. “Treating” also includes preventing an infection, or preventing a minor infection from growing into a larger infection. Even though the patient may not observe such symptoms, the microbial agents may still be present but are less metabolically active or at a reduced stage. Treating a mammal who has a diabetic foot infection to prevent future occurrences is included within the scope of “treating” as used in the present invention.

According to the method of the present invention, the oxazolidinones can be used either individually or in combination with each other. Further, they can be used in combination with other antibacterial agents or antibiotic compounds which are being administered by oral, intravenous, parenteral, or topical administration. The term “other antibiotic” or “second antibiotic” refers to an antibacterial agent other than the compound of the present invention. This includes but is not limited to, aminoglycoside, cephalosporin, macrolide, penem, quinolones, sulfas, tetracycline and other antibiotics such as, Amikacin, Gentamicin, Spectinomycin, Tobramycin, Imipenem, Meropenem, Cefadroxil, Cefazolin, Cephalexin, Cefaclor, Cefotetan, Cefoxitin, Cefprozil, Cefuroxime, Loracarbef, Cefdinir, Cefixime, Cefoperazone, Cefotaxime, Cefpodoxime, Ceftazidime, Ceftibuten, Ceftozoxime, Ceftriaxone, Cefepime, Azithromycin, Clarithromycin, Dirithromycin, Penicillin G. Cloxacillin, Dicloxacillin, Nafcillin, Oxacillin, Amnoxicillin, Amoxicillin, Ampicillin, Meziocillin, Piperacillin, Nalidixic Acid, Ciprofloxacin, Enoxacin, Lomefloxacin, Norfloxacin, Ofloxacin, Levofloxacin, Sparfloxacin, Alatrofloxacin, Gatifloxacin, Moxifloxacin, Trimethoprim, Sulfisoxazole, Sulfamethoxazole, Doxycycline, Minocycline, Tetracycline, Aztreonam, Chloramphenicol, Clindamycin, Quinupristin, Fosfomycin, Metronidazole, Nitrofurantoin, Rifampin, Trimethoprim, and Vancomycin. All of them are known. They can be either obtained commercially or be prepared according to the references cited in PHYSICIANS' DESK REFERENCE, the 53rd Edition (1999) and the U.S.F.D.A.'s Orange book.

It is preferred that these other antibiotics are administered to deliver 1-mg/kg/day for an adult. In addition, the oxazolidinone can be used with non-antibiotic agents in treating diabetic foot infections. One possible advantage of this aspect of the invention is that relatively smaller amounts of the active agents can be used to obtain a high level of antibacterial activity. The invention allows high levels of antibacterial effect to be achieved using relatively small amounts of active agent than when compared with the individual antibacterial components used in the invention. This advantage can be particularly beneficial in patients also having neutropenia, such as patients suffering from leukemia or lymphoma.

In addition, the combined use of the oxazolidinone compound, particularly linezolid, with other antibacterial agents such as a cephalosporin, aminoglycoside, or penem provides a new broad spectrum of antibacterial activity. The methods demonstrate antibacterial activity against a broad spectrum of gram-positive and gram-negative infectious agents, including gram-negative aerobes and anaerobes. Moreover, the invention allows more rapid and complete elimination of difficult to treat gram-positive infections, particularly in difficult to penetrate regions of the body where local conditions are unfavorable toward eliminating the microorganism by a single antibacterial agent. These combinations can be administered in accordance with the method of the invention. The method provides for treating a diabetic foot infection by administering, singly or together, oxazolidinone, cephalosporin, aminoglycoside, or penem active agents. The active agents may, but need not, be admixed to provide a mixture having therapeutic activity. Alternatively, the active agents may be administered separately, or two of the three active agents may be combined and administered separately of the third active agent.

The exact dosage and frequency of administration depends on the particular oxazolidinone used, the severity of the condition being treated, the age, weight and general physical condition of the particular patient, and other medication the particular patient may be taking as is well known to those skilled in the art and can be more accurately determined by measuring the blood level or concentration of the oxazolidinone in the patient's blood and/or the patient's response to the particular treatment administered. If the treatment is in combination with oral, parenteral, or intravenous administration of other medicaments, the blood level or concentration of the other medicaments(s) in the patient's blood can also be measured.

Without further elaboration, it is believed that one skilled in the art can, using the preceding description, practice the invention to its fullest extent. The following detailed examples describe how to prepare the various compounds and/or perform the various processes of embodiments of the invention and are to be construed as merely illustrative, and not limitations of the preceding disclosure in any way whatsoever. Those skilled in the art may recognize appropriate variations from the procedures both as to reactants and as to reaction conditions and techniques.

### EXAMPLES

### Example 1

### (S)-N-[[3-[3-fluoro-4-(4-morpholinyl)phenyl]-2-oxo-5-oxazolidinyl]methyl]acetamide.

(S)-N-[[3-[3-Fluoro-4-(4-morpholinyl)phenyl]-2-oxo-5-oxazolidinyl]methyl]acetamide (linezolid) is known. See U.S. Pat. No. 5,688,792, (Example 5).

### Example 2

### Treatment of a Human With a Diabetic Foot Infection by Oral and/or Intravenous Administration of an Oxazolidinone

Male and female patients at least 18 years old with a history of diabetes mellitus and a diabetic foot infection, were treated with an oral (PO) and/or an intravenous (IV) preparation of linezolid in an open-label, comparator controlled study A total of 371 patients were randomized on a 2:1 ratio between linezolid (248 patients) and aminopenicillins (123 patients). The clinically evaluable population comprised 317 patients, most of whom were male and white with a mean age of 63 years old. Patients with critical limb ischemia were excluded, but those with osteomyelitis could be included. The hospitalized patients and outpatients received either: 1) a preparation of 600 mg of linezolid administered (IV or PO) two times daily; 2) a preparation of 1.53 g of ampicillin-sulbactam administered (IV) four times daily; or 3) a preparation of 500-875 mg of ampicillin-clavulanate administered (PO) three to four times daily. Treatment was for 7-28 days with the mean treatment duration shown below in Table 1. Patients could be switched to PO therapy at the investigator's discretion.

TABLE 1
Mean (±SD) Treatment Duration (days)

| | |
|---|---|
| Treatment | Antibiotic |

| Type | Linezolid | Amino/β-LI |
|---|---|---|
| IV | 7.8 ± 5.5 | 10.4 ± 5.7 |
| PO | 15.9 ± 7.4 | 15.0 ± 7.8 |
| Total | 17.2 ± 7.9 | 16.5 ± 7.9 |

The most common baseline pathogens were S. aureus (158 isolates, 31 of which were methicillin-resistant S. aureus (MRSA), coagulase-negative staphylococci (65), enterococci (60) and Streptococcus agalactiae (52). To cover methicillin-resistant S. aureus in the aminopenicillin regimen, IV vancomycin (1 g every 12 h or per dosing guidelines) was added to the amino/β-LI treated patients with MRSA isolated from the study wound. To cover possible resistant gram-negative rods in either regimen, IV aztreonam (1-2 g, IV, every 8-12 h) was permitted in both treatment arms for suspected/documented gram-negative pathogens. This is shown below in Table 2.

TABLE 2
Added IV Antibiotics

| | Linezolid | Amino/β-LI |
|---|---|---|
| Vancomycin | 1 patient | 5 patients |
| | (0.4% of patients) | (9.6% of patients) |
| Aztreonam | 12 patients | 3 patients |
| | (5.0% of patients) | (2.5% of patients) |

241 patients received linezolid, 120 patients received amino/β-LI; and 10 patients received no treatment and were non-evaluable.

The efficacy and safety of 7-28 days of treatment with linezolid was compared with treatment with aminopenicillin agents commonly used for diabetic foot infection (DFI), ampicillin/sulbactam (IV) and/or amoxicillin/clavulanate (PO). Infections were defined by clinical signs and symptoms and categorized as cellulites, deep soft tissue infections, infected ulcer, septic arthritis, paronychia, abscess or osteomyelitis. Patients in the two arms were comparable at baseline in their demographic characteristics, clinical findings, and laboratory test results. The most common types (sometimes combined) of infections were: ulcers (78%); cellulitis (45%); deep soft tissue (15%); paronychia (6%). Most patients in both arms were treated as outpatients (66%) with only oral therapy (73%) and with a single agent (87%). Clinical characteristics of the infected sites at baseline were similar in the treatment groups; most common were tenderness (97%), induration (95%), local warmth (92%), non-purulent drainage (82%) and erythema (47%).

Global evaluation of the two arms at end of therapy and at follow-up (15-21 days later) are shown in the tables below. Debridement and other surgical procedures (short of complete resection/amputation) were allowed and wounds were off-loaded (mechanical stress on the wounded area was avoided) as needed. There were no significant differences in adverse events in the two treatment arms.

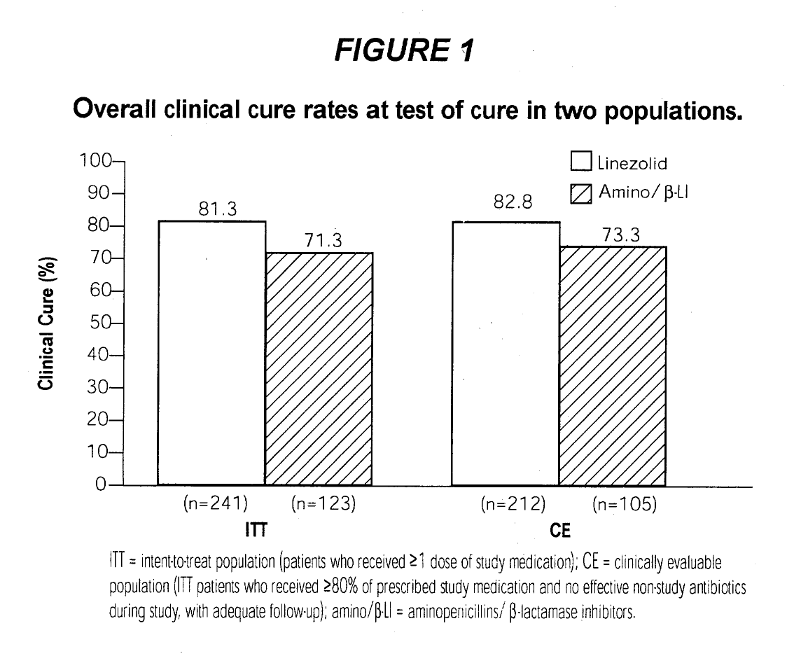

Clinical cure rates for linezolid and amino/β-LI-treated patients in the intent-to-treat (ITT) and clinically evaluable (CE) populations were comparable (see FIG. 1).

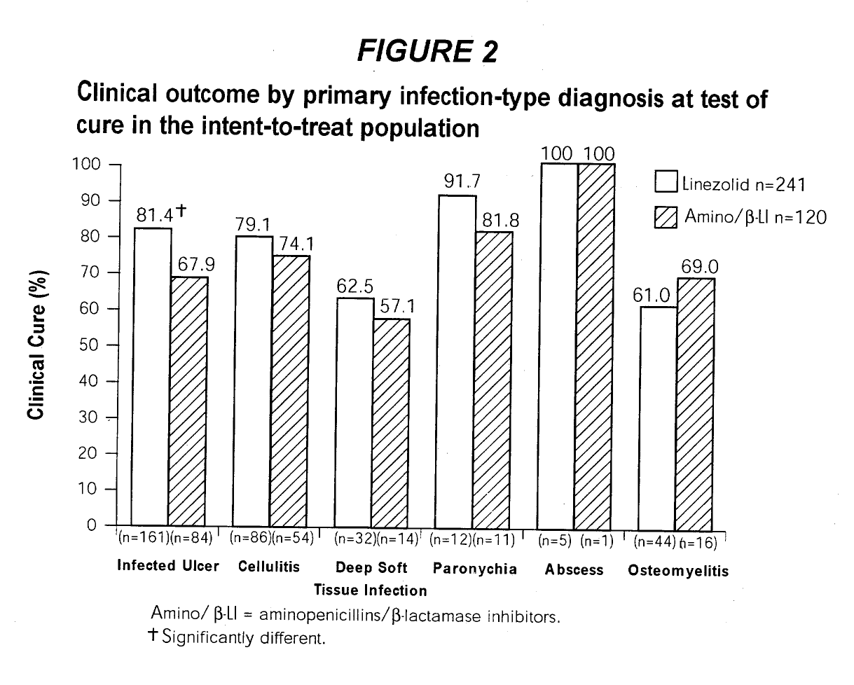

For infected ulcers, significantly more linezolid-treated patients than amino/β-LI-treated patients were clinically cured (81.4% vs. 67.9%; 95% confidence interval (CI): 4.5, 25.7) (see FIG. 2).

In patients without osteomyelitis, linezolid-treated patients had a significantly higher clinical cure rate than those who received amino/β-LI (86% vs. 71%; 95% CI: 4.5, 25.7). In patients with osteomyelitis (n=60), clinical cure rates were comparable (61.0% vs. 69.0%, respectively, see FIG. 2). No significant differences were observed between treatment groups in clinical outcome by baseline pathogen, except for a significantly higher clinical cure rate observed for linezolid for S. agalactiae (see Table 3).

TABLE 3
Clinical Outcome by Baseline Pathogen (MITT Population)

| | Linezolid | Amino/β-LI | |
|---|---|---|---|
| Pathogen | n/N % | n/N (%) | 95% CI |
| Staphylococcus aureus (MSSA) | 50/67 (75) | 28/39 (72) | −14.7, 20.4 |
| Staphylococcus aureus (MRSA | 13/18 (72) | 4/7 (57) | −27.0, 57.2 |
| Staphylococcus agalactiae | 26/31 (84) | 9/18 (60) | 7.4, 60.4 |
| Coagulase-negative | 31/35 (89) | 17/19 (90) | −18.3, 16.5 |
| staphylococci | | | |
| Enterococcus spp. | 23/34 (68) | 13/17 (76) | 34.4, 16.8 |

MITT = modified intent to treat (intent-to-treat patients with a baseline pathogen identified);
amino/β-LI = aminopenicillins/β-lactamase inhibitors;
n/N = number of patients responding to treatment/number of patients treated;
CI = confidence interval;
MSSA = methicillin-sensitive Staphylococcus aureus;
MRSA = methicillin resistant Staphylococcus aureus.

As summarized below in Table 4, the microbiological success rates (microbiologically evaluable (ME) patients included those CE patients with a confirmed baseline gram-positive pathogen susceptible to study medication) between these two types of treatment were comparable: 72.2% in linezolid-treated versus 63.0% in amino/β-LI-treated patients (95% CI: −5.5, 23.8). None of the isolated pathogens was resistant to linezolid at baseline or developed resistance during the trial. Resistance to ampicillin/sulbactam was documented in 1 S. aureus isolate.

Resistance to amoxicillin/clavulanate was noted in 7 S. aureus isolates and in 4 S. epidermidis isolates.

TABLE 4
Overall Clinical and Microbiological Efficacy.

| | | | Amino/β-LI | |
|---|---|---|---|---|
| | | Linezolid | Comparator | |
| | | (% Efficacy | (% Efficacy | |
| | Total No. | of the | of the | |
| | Evaluatable | Evaluatable | Evaluatable | |
| Parameter | Patients | Population) | Population) | 95% C.I. |
| Clinical | 317 | 83% | 73% | 0.006, 0.197 |
| Efficacy | | | | |
| (Clinic. | | | | |
| Eval. Pop.) | | | | |
| Microbiol. | 212 | 72% | 63% | −0.055, |
| Efficacy | | | | 0.238 |
| (Micro. | | | | |
| Eval. Pop) | | | | |

From this large, randomized trial of treating DFI, linezolid (mostly given alone, PO/orally, to outpatients) was at least as effective overall as amino/β-LI with respect to clinical and micro-biological outcomes in treating DFI, and was clinically superior for treating infected ulcers and non-osteomyelitis cases. Linezolid, therefore offers an additional IV or PO agent against potentially resistant gram-positive organisms and has a role as, an alternative to amino/β-LI therapy in treating DFI.

### Example 3

### A Human Who has a Diabetic Foot Infection is Treated by Parenteral Administration of an Oxazolidinone

A 64-year-old 70 kilo female with a history of diabetes and infected foot ulcerations, also known as diabetic foot, is treated with a parenteral preparation of 600 mg (S)-N-[[3-[3-fluoro-4-[4-(morpholinyl)phenyl]-2-oxo-5-oxazolidinyl]methyl]acetamide. The preparation is administered two times daily for 7-28 days. The patient is evaluated by clinical observations and with x-rays for osteomyelitis, including with a “probe-to bone” test and a bone biopsy if the patient has an open wound. Following the course of oxazolidinone treatment, tissue destruction in the ulcerated area has visibly ceased and tissue repair has begun as evidenced by lack of serous-fluid “weeping” and reduction in swelling.

Accordingly, the oral, parenteral, or intravenous administration of linezolid provides promising activity in the treatment of diabetic foot infections. The method can be useful in the treatment of diabetic foot infections, including infections caused by resistant stains with reduced susceptibility to other antibiotics.

The foregoing detailed description is given for clearness of understanding only, and no unnecessary limitations should be understood therefrom, as modifications within the scope of the invention may become apparent to those skilled in the art.

What is claimed:

1. A method of treating a diabetic foot infection in a mammal comprising orally administering to the mammal a pharmaceutically effective amount of an oxazolidinone antibiotic.

2. The method of treating a diabetic foot infection according to claim 1 wherein the antibiotic is in the form of a pharmaceutical formulation.

3. The method of treating a diabetic foot infection according to claim 1 wherein the mammal is selected from the group consisting of a human, a livestock animal, and a companion animal.

4. The method of treating a diabetic foot infection according to claim 3 wherein the mammal is a human.

5. The method of treating a diabetic foot infection according to claim 2 wherein the pharmaceutical formulation comprises plain or coated tablets, capsules, lozenges, powders, solutions, suspensions, emulsions, syrups, or combinations thereof.

6. The method of treating a diabetic foot infection according to claim 2 wherein the pharmaceutical formulation has a pharmaceutically-effective amount of oxazolidinone from about 200 mg to about 900 mg.

7. The method of treating a diabetic foot infection according to claim 6 wherein the pharmaceutical formulation has a pharmaceutically-effective amount of oxazolidinone from about 500 mg to about 700 mg.

8. The method of treating a diabetic foot infection according to claim 7 wherein the pharmaceutical formulation has a pharmaceutically-effective amount of oxazolidinone of about 600 mg.

9. The method of treating a diabetic foot infection according to claim 1 wherein the method further comprises administering to the mammal a second antibiotic.

10. The method of treating a diabetic foot infection according to claim 1 wherein the method further comprises administering to the mammal a non-antibiotic agent.

11. The method of treating a diabetic foot infection according to claim 9 wherein the second antibiotic is selected from the group consisting of an aminoglycoside, a cephalosporin, a macrolide, a penem, a quinolone, a sulfa, a tetracycline, and combinations thereof.

12. The method of treating a diabetic foot infection according to claim 9 wherein the second antibiotic is administered orally, parenterally, intravenously, or topically to administer 1-10 mg/kg per day for an adult.

13. The method of treating a diabetic foot infection according to claim 1 wherein said method is carried out for one to 60 days.

14. The method of treating a diabetic foot infection according to claim 1 wherein the pharmaceutical formulation is administered from 2 to 4 times daily.

15. The method of treating a diabetic foot infection according to claim 1 wherein the diabetic foot infection is manifested by purulent or non-purulent drainage or discharge, erythema, fluctuance, heat or localized warmth, pain or tenderness to palpation, an inflamed, reddened, swollen, indurated or tender area on the foot under broken or unbroken skin and which may be coupled with a fever.

16. The method of treating a diabetic foot infection according to claim 1 wherein the infection is caused by staphylococci, streptococci, enterococci, or combinations thereof.

17. The method of treating a diabetic infection according to claim 16 wherein the infection is caused by staphylococci.

18. The method of treating a diabetic foot infection according to claim 1 wherein the infection is caused by a resistant strain of bacteria selected from the group consisting of methicillin-resistant Staphylococcus aureus (MRSA), vancomycin resistant Enterococci (VRE), glycopeptide-intermediate Staphylococcus aureus (GISA) and vancomycin resistant Staphylococcus aureus (VISA), and combinations thereof.

19. The method of treating a diabetic foot infection according to claim 1 wherein the compound is of the formula: 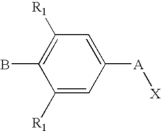
or a pharmaceutically acceptable salt thereof wherein:
A is a structure i, ii, iii, or iv 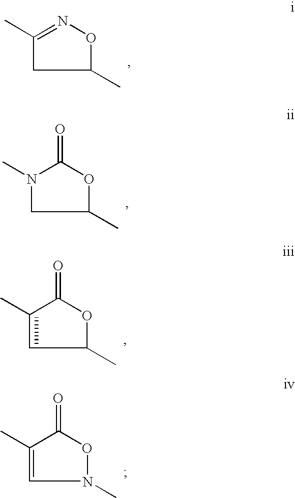
B is selected from cycloalkyl, substituted cycloalkyl, cycloalkenyl, substituted cycloalkenyl, aryl, substituted aryl, het and substituted het, or
B and one R₁ together, with the phenyl carbon atoms to which B and the one R₁ are bonded, form a het, the het optionally being a substituted het;
X is a group selected from —CH₂—NH—C(O)—R₂, —CH₂—R₂, and —CH₂—Y—R₂;
Y is O, S, or —NH—;
R₁ is independently selected from H, alkyl, alkoxy, amino, NO₂, CN, halo, substituted alkyl, substituted alkoxy, and substituted amino; and
R₂ is independently selected from H, —OH, amino, alkyl, substituted alkyl, alkoxy, substituted alkoxy, alkenyl, substituted alkenyl, cycloalkyl, substituted cycloalkyl, cycloalkenyl, substituted cycloalkenyl, het, substituted het, aryl, and substituted aryl.

20. The method of treating a diabetic foot infection according to claim 19 wherein the oxazolidinone is of the formula: 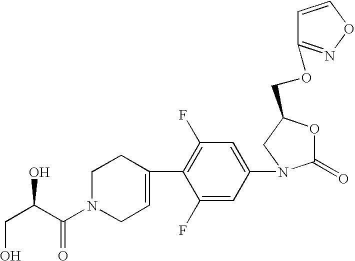
or a pharmaceutically acceptable salt thereof.

21. The method of treating a diabetic foot infection according to claim 19, wherein the oxazolidinone is of the formula: 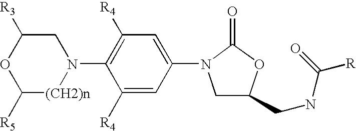
or is a pharmaceutically acceptable salt thereof, wherein:
n is 0, 1, or 2;
R is selected from the group consisting of:
hydrogen;
C₁-C₈ alkyl optionally substituted with one or more substituents selected from the group consisting of F, Cl, hydroxy, C₁-C₈ alkoxy, C₁-C₈ acyloxy, or —CH₂-phenyl;
C₃-C₆ cycloalkyl;
amino;
C₁-C₈ alkylamino;
C₁-C₈ dialkylamino; or
C₁-C₈ alkoxy;
R³ at each occurrence is independently selected from the group consisting of H, CH₃, CN, CO₂H, CO₂R, and (CH₂)ₘR⁶, wherein m is 1 or 2;
R⁴ at each occurrence is independently selected from the group consisting of H, F, and Cl;
R⁵ is H or CH₃;
R⁶ is selected from the group consisting of H, OH, OR, OCOR, NH₂, NHCOR, and N(R⁷)₂; and
R⁷ at each occurrence is independently selected from the group consisting of H, p-toluensulfonyl, and C₁-C₄ alkyl optionally substituted with one or more substituents selected from the group consisting of Cl, F, OH, C₁-C₈ alkoxy, amino, C₁-C₈ alkylamino, and C₁-C₈ dialkylamino.

22. The method of treating a diabetic foot infection according to claim 1, wherein the oxazolidinone is of the formula: 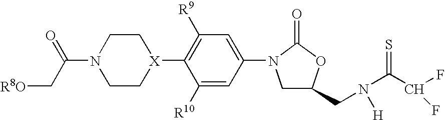
or is a pharmaceutically acceptable salt thereof, wherein:
X is N or CH;
R⁹ and R₁₀ are independently H or F; and
R⁸ is H, benzyl, or —C(═O)C₁₋₄alkyl.

23. The method of treating a diabetic foot infection according to claim 22 wherein the oxazolidinone compound is 2,2-difluoro-N-({(5S)-3-[3-fluoro-4-(4-glycoloylpiperazin-1-yl)phenyl]-2-oxo-1,3-oxazolidin-5-yl}methyl)ethanethioamide.

24. The method of treating a diabetic foot infection according to claim 21 wherein the oxazolidinone compound is (S)-N-[[3-[3-fluoro-4-(4-morpholinyl)phenyl]-2-oxo-5-oxazolidinyl]methyl]acetamide.

25. A method of treating a diabetic foot infection in a mammal comprising parenterally administering to the mammal a pharmaceutically effective amount of an oxazolidinone antibiotic.

26. The method of treating a diabetic foot infection according to claim 25 wherein the parenterally administering is intravenously administering.

27. The method of treating a diabetic foot infection according to claim 25 wherein the oxazolidinone antibiotic is in the form of a pharmaceutical formulation.

28. The method of treating a diabetic foot infection according to claim 25 wherein the mammal is selected from the group consisting of a human, a livestock animal, and a companion animal.

29. The method of treating a diabetic foot infection according to claim 28 wherein the mammal is a human.

30. The method of treating a diabetic foot infection according to claim 27 wherein the pharmaceutical formulation comprises solutions, suspensions, emulsions, syrups, or combinations thereof.

31. The method of treating a diabetic foot infection according to claim 25 wherein the pharmaceutical formulation has a pharmaceutically-effective amount of oxazolidinone from about 200 mg to about 900 mg.

32. The method of treating a diabetic foot infection according to claim 31 wherein the pharmaceutical formulation has a pharmaceutically-effective amount of oxazolidinone from about 500 mg to about 700 mg.

33. The method of treating a diabetic foot infection according to claim 32 wherein the pharmaceutical formulation has a pharmaceutically-effective amount of oxazolidinone of about 600 mg.

34. The method of treating a diabetic foot infection according to claim 25 wherein the method further comprises administering to the mammal a second antibiotic.

35. The method of treating a diabetic foot infection according to claim 25 wherein the method further comprises administering to the mammal a non-antibiotic agent.

36. The method of treating a diabetic foot infection according to claim 34 wherein the-second antibiotic is selected from the group consisting of an aminoglycoside, a cephalosporin, a macrolide, a penem, a quinolone, a sulfa, a tetracycline, and combinations thereof.

37. The method of treating a diabetic foot infection according to claim 34 wherein the second antibiotic is administered orally, parenterally, intravenously, or topically to administer 1-10 mg/kg per day for an adult.

38. The method of treating a diabetic foot infection according to claim 25 wherein said method is carried out for one to 60 days.

39. The method of treating a diabetic foot infection according to claim 25 wherein the pharmaceutical formulation is administered from 2 to 4 times daily.

40. The method of treating a diabetic foot infection according to claim 25 wherein the diabetic foot infection is manifested by purulent or non-purulent drainage or discharge, erythema, fluctuance, heat or localized warmth, pain or tenderness to palpation, an inflamed, reddened, swollen, indurated or tender area on the foot under broken or unbroken skin and which may be coupled with a fever.

41. The method of treating a diabetic foot infection according to claim 25 wherein the infection is caused by staphylococci, streptococci, enterococci, or combinations thereof.

42. The method of treating a diabetic infection according to claim 41 wherein the infection is caused by staphylococci.

43. The method of treating a diabetic foot infection according to claim 25 wherein the infection is caused by a resistant strain of bacteria selected from the group consisting of methicillin-resistant Staphylococcus aureus (MRSA), vancomycin resistant Enterococci (VRE), glycopeptide-intermediate Staphylococcus aureus (GISA) and vancomycin resistant Staphylococcus aureus (VISA), and combinations thereof.

44. The method of treating a diabetic foot infection according to claim 25 wherein the oxazolidinone is of the formula: 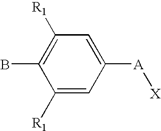
or a pharmaceutically acceptable salt thereof wherein:
A is a structure i, ii, iii, or iv 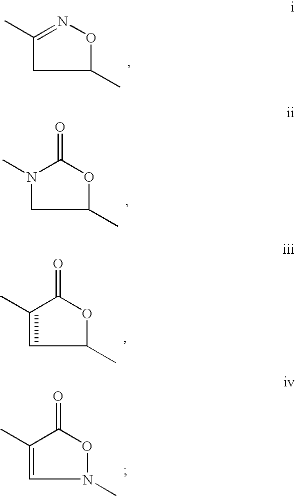
B is selected from cycloalkyl, substituted cycloalkyl, cycloalkenyl, substituted cycloalkenyl, aryl, substituted aryl, het and substituted het, or
B and one R₁ together, with the phenyl carbon atoms to which B and the one R₁ are bonded, form a het, the het optionally being a substituted het;
X is a group selected from —CH₂—NH—C(O)—R₂, —CH₂—R₂, and —CH₂—Y—R₂;
Y is O, S, or —NH—,
R₁ is independently selected from H, alkyl, alkoxy, amino, NO₂, CN, halo, substituted alkyl, substituted alkoxy, and substituted amino; and
R₂ is independently selected from H, —OH, amino, alkyl, substituted alkyl, alkoxy, substituted alkoxy, alkenyl, substituted alkenyl, cycloalkyl, substituted cycloalkyl, cycloalkenyl, substituted cycloalkenyl, het, substituted het, aryl, and substituted aryl.

45. The method of treating a diabetic foot infection according to claim 44 wherein the oxazolidinone is of the formula II:

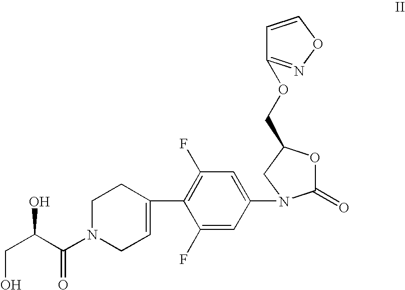

46. The method of treating a diabetic foot infection according to claim 25 wherein the oxazolidinone is of the formula: 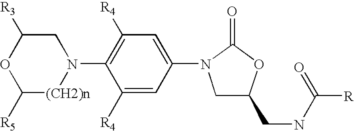
or is a pharmaceutically acceptable salt thereof, wherein:
n is 0, 1, or 2;
R is selected from the group consisting of:
hydrogen;
C₁-C₈ alkyl optionally substituted with one or more substituents selected from the group consisting of F, Cl, hydroxy, C₁-C₈ alkoxy, C₁-C₈ acyloxy, or —CH₂-phenyl;
C₃-C₆ cycloalkyl;
amino;
C₁-C₈ alkylamino;
C₁-C₈ dialkylamino; or
C₁-C₈ alkoxy;
R³ at each occurrence is independently selected from the group consisting of H, CH₃, CN, CO₂H, CO₂R, and (CH₂)ₘR⁶, wherein m is 1 or 2;
R⁴ at each occurrence is independently selected from the group consisting of H. F, and Cl;
R⁵ is H;
R⁶ is selected from the group consisting of H, OH, OR, OCOR, NH₂, NHCOR, and N(R⁷)₂; and
R⁷ at each occurrence is independently selected from the group consisting of H, p-toluensulfonyl, and C₁-C₄ alkyl optionally substituted with one or more substituents selected from the group consisting of Cl, F, OH, C₁-C₈ alkoxy, amino, C₁-C₈ alkylamino, and C₁-C₈ dialkylamino.

47. The method of treating a diabetic foot infection according to claim 25, wherein the oxazolidinone is of the formula: 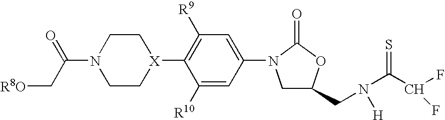
or is a pharmaceutically acceptable salt thereof, wherein:
X is N or CH;
R⁹ and R¹⁰ are independently H or F; and
R⁸ is H, benzyl, or —C(═O)C₁₋₄alkyl.

48. The method of treating a diabetic foot infection according to claim 47 wherein the oxazolidinone compound is 2,2-difluoro-N-({(5S)-3-[3-fluoro-4-(4-glycoloylpiperazin-1-yl)phenyl]-2-oxo-1,3-oxazolidin-5-yl} methyl)ethanethioamide.

49. The method of treating a diabetic foot infection according to claim 46 wherein the oxazolidinone compound is (S)-N-[[3-[3-fluoro-4-(4-morpholinyl)phenyl]-2-oxo-5-oxazolidinyl]methyl]acetamide.

50. The use of a composition comprising a pharmaceutically effective amount of an oxazoldinone compound for the manufacture of a medicament for preventing or treating a diabetic foot infection.

51. The use of a composition according to claim 50 wherein the oxazolidinone compound is of the formula: 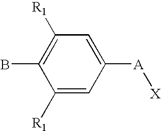
or a pharmaceutically acceptable salt thereof wherein:
A is a structure i, ii, iii, or iv; 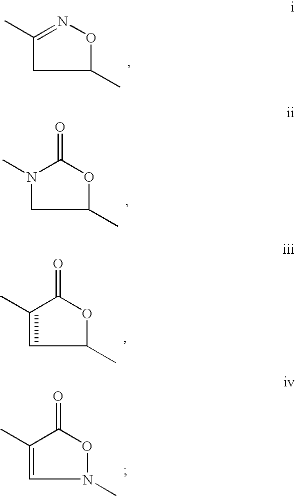
B is selected from cycloalkyl, substituted cycloalkyl, cycloalkenyl, substituted cycloalkenyl, aryl, substituted aryl, het and substituted het, or
B and one R₁ together, with the phenyl carbon atoms to which B and the one R₁ are bonded, form a het, the het optionally being a substituted het;
X is a group selected from —CH₂—NH—C(O)—R₂, —CH₂—R₂, and —CH₂—Y—R₂;
Y is O, S, or —NH—;
R₁ is independently selected from H, alkyl alkoxy, amino, NO₂, CN, halo, substituted alkyl, substituted alkoxy, and substituted amino; and
R₂ is independently selected from H, —OH, amino, alkyl, substituted alkyl, alkoxy, substituted alkoxy, alkenyl, substituted alkenyl, cycloalkyl, substituted cycloalkyl, cycloalkenyl, substituted cycloalkenyl, het, substituted het, aryl, and substituted aryl.

52. The use of a composition according to claim 50 wherein the oxazolidinone compound is of the formula: 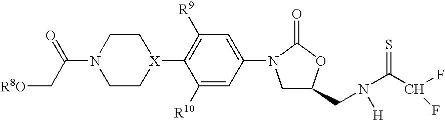
or is a pharmaceutically acceptable salt thereof, wherein:
X is N or CH;
R⁹ and R¹⁰ are independently H or F; and
R⁸ is H, benzyl, or —C(═O)C₁₋₄alkyl.
